(19) United States
(12) Patent Application Publication
(10) Pub. No.: US 2009/0023670 A1
(43) Pub. Date: Jan. 22, 2009

(54) Regulation of Transgene Expression by RNA Interference

(76) Inventor: Magdolna G. Sebestyen, Madison, WI (US)

Correspondence Address: MIRUS CORPORATION, 505 SOUTH ROSA RD, MADISON, WI, 53719, US

(21) Appl. No.: 11/750,407

(22) Filed: May 18, 2007

Related U.S. Application Data
(60) Provisional application No. 60/801,897, filed on May 19, 2006.

(51) Int. Cl. A61K 31/7088; C12N 5/06; C12N 15/00; A61P 7/00

(52) U.S. Cl. 514/44; 435/375; 435/320.1

(57) ABSTRACT

Expression of transgenes delivered into a host organism cells can be regulated by RNA effector molecules delivered to or present in the host organism cells. Regulation can be mediated by delivery of RNA interference inducing molecules that target the transgene mRNA or by incorporating engineered RNA effector binding sites into the transgene. Temporary or long term regulation of expression can be achieved depending on the nature and dosing of RNA effector.

### CROSS-REFERENCE TO RELATED APPLICATIONS

This application claims the benefit of U.S. Provisional Application No. 60/801,897, filed May 19, 2006.

### BACKGROUND OF THE INVENTION

Hormones, growth factors, coagulation factors, antibodies and other immunoregulatory molecules, enzymes and a complex array of other proteins constantly circulate in the serum. The proper balance of these components is required to ensure the body's homeostasis. Both the lack of and the overproduction of these factors can lead to disease. When a chronic illness is associated with the lack or insufficient production of a serum protein, the patient may benefit from gene therapy. Serum proteins can be produced and secreted into the blood stream by delivering their genes to ectopic sites. Gene transfer into skeletal muscle is especially attractive given the large, easily accessible cell mass and its intricate connection with the blood supply. Muscle is therefore a highly desirable target organ for several gene therapy applications including the production of secreted proteins. However, independently of the site of protein production and secretion, it is preferable to maintain a proper balance of serum protein levels.

Chronic anemia and its treatment with erythropoietin (EPO) is an example of serum protein insufficiency and its treatment with gene therapy. EPO plays a central role in the regulation of red blood cell production by controlling the proliferation, differentiation and survival of erythroid progenitors in the bone marrow. Patients suffering from various chronic diseases, such as progressive kidney failure, AIDS, or cancer, often develop anemia that can be alleviated by EPO replacement therapy (Weiss et al. 2005). Most of these patients are currently treated by the frequent injection of recombinant human EPO (huEPO) protein. Gene therapy can provide a longer-term solution to these patients.

One of the challenges of gene replacement therapy, such as EPO-based gene therapy for anemia, is the regulation of gene expression. The overexpression of EPO leads to abnormal levels of erythropoiesis, resulting in polycythemia. This condition can cause serious health problems and may even be fatal. Thus, controlling serum EPO levels after gene delivery has been an important focus of EPO-based gene therapy research. Several regulatable expression systems have been developed to permit transcription only in the presence of a small-molecule ligand such as mifepristone (Nordstrom 2003), doxycyclin (Lamartina et al. 2002, Chenuaud et al. 2004) or the “dimerizer” agent, rapamycin (Rivera et al. 2005). All these approaches involve the long-term administration of a drug, which itself may have side effects. Even more problematic for longer term correction is that these systems utilize chimeric transactivators to regulate transcription. These protein-based regulators can elicit an immune response resulting in the rejection of all expressing cells (Favre et al. 2002, Latta-Mahieu et al. 2002). These approaches are also based on the delivery of a high copy number of the transgene followed by the regulation of transcription. A safer alternative, for EPO expression, involves controlling EPO expression via a hypoxia response element in the enhancer (Binley et al. 2002). However, due to the complexity of the mechanisms that normally regulate physiological EPO expression, mimicking these mechanisms may be quite challenging.

While many gene therapy efforts aim to increase transgene delivery and expression efficiency levels, there are situations, such as that described above, when the ability to down-regulate transgene expression is important. Over-expression of some growth factors, hormones and other biologically active proteins can cause harmful effects, just as their lack of their expression can cause problems. The ability to attenuate transgene expression when necessary would increase the safety of gene therapy approaches. The invention disclosed here provides tools to regulate expression of transgenes.

### SUMMARY OF THE INVENTION

Described herein are methods and compositions for regulating in vivo protein production from a transgene. The methods and compositions work with the naturally occurring endogenous RNA interference pathways present in mammalian cells. RNA effector (hereafter “RNA effector” or “effector”). The described system can attenuate expression from a transgene which has an RNA effector binding site.

In a preferred embodiment, an RNA effector, and especially a synthetic effector, can contain, or be designed to contain, sequence that is complementary to a sequence that naturally occurs in the transgene such that the effector binds to the sequence in the transgene mRNA and inhibits expression of the transgene. The sequence in the transgene may or may not be a target site for a naturally occuring miRNA. The effector is delivered to cells in vivo in which the transgene is expressed. The effector can be delivered prior to delivery of the transgene, co-delivered with the transgene, or delivered subsequent to transgene delivery.

In a preferred embodiment, an RNA effector, and especially a synthetic effector, can contain, or be designed to contain, sequence that is complementary to an ectopic effector binding site that is engineered into the transgene such that the effector binds to the effector binding site in the transgene mRNA and inhibits expression of the transgene. The ectopic effector binding site can be a known siRNA or miRNA binding site or it can be an artificial sequence that is engineered into the transgene. For artificial binding sites, cognate effectors are readily created using techniques standard in the art. The effector is delivered to cells in vivo in which the transgene is expressed. The effector can be delivered prior to delivery of the transgene, co-delivered with the transgene, or delivered subsequent to transgene delivery.

In a preferred embodiment, an ectopic effector binding site can be engineered into the transgene such that a known miRNA in the target cell binds to the effector binding site in the transgene mRNA and inhibits expression of the transgene. The transgene is delivered to cells in vivo in which the endogenous known miRNA is expressed.

In a preferred embodiment, an expression cassette which expresses a transgene is engineered to contain an ectopic RNA effector binding site. The binding of a cognate effector to the effector binding site results in decreased translation of mRNA transcribed from the transgene. The RNA effector can be naturally occurring in a cell to which the transgene is delivered. Alternatively, a synthetic effector can be delivered to a cell in vivo in which the transgene is expressed. The effector can be delivered prior to delivery of the transgene, co-delivered with the transgene, or delivered subsequent to transgene delivery.

In a preferred embodiment, a transgene expression cassette is described which encodes a transcript that contains one or more ectopic RNA effector binding sites. The effector binding sites can be located in the 5′ untranslated region (UTR), coding region, or 3′ UTR of the transgene transcript. A preferred location is in the 3′ UTR of the transgene transcript.

In a preferred embodiment, the effector may comprise: a naturally occurring miRNA, synthetic siRNA or miRNA, modified siRNA or miRNA, or other sequence-specific RNA interference inducing molecule. In another embodiment, an effector RNA molecule can be produced in vivo by transcription of a delivered gene that encodes an RNA effector. The delivered gene can transcribe the effector RNA from a constitutive or regulated promoter.

In a preferred embodiment, an effector is used that has a relatively short half life. By using an effector with a relatively short half-life, inhibition of gene expression by the effector decreases over time, resulting temporary inhibition of transgene expression.

In a preferred embodiment, the duration of regulation of the transgene by the effector can be extended by delivering RNA effectors to cells expressing the transgene multiple times—repeat dosing. The dosage (amount of delivered effector) and the interval between doses are dependent on the level of regulation desired and the delivery efficiency.

In a preferred embodiment, the effector is co-delivered with the transgene. Frequently, a transgenes delivered to cells in vivo by techniques known in the art initially express at a high level. Co-delivery of an effector specific for the transgene attenuates this initial high level of expression.

In a preferred embodiment, the transgene comprises a therapeutic transgene. In another embodiment the transgene comprises erythropoietin (EPO). In another embodiment the transgene comprises human EPO. Thus, the invention provides a method for controlling EPO expression, and therefore erythropoiesis, by the interaction of target-specific effector with the transcribed EPO mRNA. The described invention can be used to control EPO expression and the resulting erythropoiesis in a clinical setting in patients treated for chronic anemia by gene therapy.

Further objects, features, and advantages of the invention will be apparent from the following detailed description when taken in conjunction with the accompanying drawings.

### BRIEF DESCRIPTION OF THE FIGURES

FIG. 1. Bar graph illustrating the in vitro effect of various siRNAs on expression of human EPO transgene in HeLa cells. Statistically significant differences are marked with asterisks: * p<0.05; ** p<0.01; *** p<0.001. (n=3 wells/condition; average±SEM are shown.)

FIG. 2. Bar graph illustrating the in vivo effect of a siRNA effectors on human EPO transgene expression in mice. Statistically significant differences are marked with asterisks: * p<0.05; ** p<0.005 (n=5 animals/group; average±SEM are shown).

FIG. 3. Graph illustrating siRNA-mediated down-regulation of human EPO expression in primate skeletal muscle. HuEPO transgene and siRNA were co-delivered on day 0. Serum EPO concentrations determined at the indicated time points are shown. FIG. 4. Graph illustrating human EPO expression in immunosuppressed rhesus monkeys, with or without co-delivery of siRNA effector. Transgene and effector or control were simultaneously delivered on day 0. Effector or control were injected again on days 14 and 28. Serum EPO concentrations determined at the indicated time points are shown.

FIG. 5. Graph illustrating relative serum EPO concentrations in rhesus monkeys expressing the human EPO transgene with or without co-delivered siRNA effector. Serum EPO levels were normalized to levels in the animal initially injected with pDNA alone.

FIG. 6. Graph illustrating hematocrit levels in immunosuppressed monkeys expressing the huEPO transgene, in the presence or absence of delivered siRNA, in their limb muscles. Hematocrit (Hct) values were determined at the indicated time points.

FIG. 7. Diagram illustrating an EPO expression plasmid with 3′UTR ectopic miRNA binding sites.

FIG. 8. Graph illustrating miRNA-based inhibition of a rhesus EPO (rhEPO) transgene in mice. Statistically significant differences on Day 7 post-injection are marked with asterisks: ** p<0.005; **** p<0.0001 (n=4 animals/group; average±SEM are shown). By Day 21, some animals in each group had mounted an immune response against rhEPO.

FIG. 9. Graph illustrating hematocrit levels in mice expressing a rhesus EPO (rhEPO) transgene without or without ectopic hsa-mir-206-3p miRNA binding sites (p<10⁻⁵; n=4 animals/group; average±SEM are shown).

### DETAILED DESCRIPTION

Described herein are methods and compositions to take advantage of the mechanism of RNA interference in eukaryotic cells to regulate or attenuate in vivo protein production from transgenes delivered by gene therapy methods. Expression from the transgene is regulated by interaction of an RNA effector molecule with the transcribed transgene. By taking advantage of small RNA effector molecules, either delivered synthetic RNAs or endogenous miRNAs, complications caused by the host's immune response are reduced or eliminated. The transgene contains one or more sequences, effector binding sites, to which one or more effectors bind. Binding of an effector to an effector binding site results in decreased translation of the transgene transcript or degradation of the transcript.

In one embodiment, the invention uses one or more synthetic RNA effector molecules delivered to the tissue or cells to which a transgene had been delivered and expressed. The RNA effectors carry sequence motifs that are complementary to a sequence present in a mRNA produced from the transgene. The efficacy of the RNA effector with the sequence can be validated prior to delivery of the transgene of the RNA effector. Effector delivery results in down-regulation of protein production that persists for the functional duration of the RNA effector in the cell.

### RNA Interference

The RNA interference (RNAi)-mediated cleavage of messenger RNA or inhibition of mRNA translation is thought to be an evolutionarily conserved mechanism of eukaryotic cells. RNAi can function to protect an organism against the expression of an intruding foreign DNA or RNA (e.g. viral infection) or to regulate gene expression. The introduction of foreign nucleic acid into the host cell can result in the presence of long dsRNAs. These dsRNA are processed by an Rnase III-type enzyme, called Dicer (Filipowicz et al. 2005), into small, typically 21-23 base pair, double strand RNA (dsRNA) fragments with 2-nucleotide 3′ overhangs, termed siRNA.

Naturally occurring micro RNAs (miRNAs) have been identified in various organisms—including humans—and in various tissues and cell types (Moss 2002). These endogenous miRNAs are transcribed by RNA Polymerase II from small genes. The initial transcript, called pri-miRNA, folds into an imperfect hairpin structure. The pri-miRNA is then cleaved and processed by a nuclease (Drosha) inside the nucleus to produce a 65-90 nucleotide pre-miRNA that has a short hairpin structure. The pre-miRNA is transported into the cytoplasm, wherein, like dsRNA, it is further cleaved by Dicer to produce a mature ˜22-bp RNA duplex.

MiRNAs or siRNAs become associated with the RNA-induced silencing complex, RISC. This complex then targets the miRNA or siRNA to a homologous region, a target or binding site, in an mRNA. Depending on the level of complementarity between the miRNA or siRNA and the target site in the mRNA the RISC complex can cause translation inhibition or mRNA degradation.

For miRNAs, the complex binds to target sites usually located in the 3′ UTR of mRNAs that typically share only partial homology with the miRNA. A “seed region”—a stretch of about 7 consecutive nucleotides on the 5′ end of the miRNA that forms perfect base pairing with its target—plays a key role in miRNA specificity. Binding of the RISC/miRNA complex to the mRNA can lead to either the repression of protein translation or cleavage and degradation of the mRNA. Recent data indicate that mRNA cleavage happens preferentially if there is perfect homology along the whole length of the miRNA and its target instead of showing perfect base-pairing only in the seed region (Pillai et al. 2007).

### Effector Molecules

As used herein, an “RNA effector” (also RNA effector molecule, effector molecule, or effector) is a molecule capable inducing RNA interference through interaction with the RNA interference pathway machinery of mammalian cells to degrade or inhibit translation of messenger RNA (mRNA) transcripts of a transgene in a sequence specific manner. The two primary effector molecules in RNAi are small (or short) interfering RNAs (siRNAs) and micro RNAs (miRNAs). However, other polynucleotides have been shown to mediate RNA interference. RNA effectors may be selected from the group comprising: siRNA, microRNA, interfering RNA or RNAi, double-strand RNA (dsRNA), short hairpin RNA (shRNA), and expression cassettes encoding RNA capable if inducing RNA interference. An RNA effector molecule can be an endogenous RNA such as an endogenous miRNA. An RNA effector can also be a synthetic siRNA or miRNA or other RNA interference inducing polynucleotide.

Small RNA molecules that are effectors of RNA interference are typically less than 50-65 nucleotides in length. SiRNA comprises a double stranded structure typically containing 15-50 nucleotides per strand and preferably about 19 to about 25 nucleotides per strand or about 22 nucleotides per strand and having a nucleotide sequence identical (perfectly complementary) or nearly identical (partially complementary) to the coding region of a target mRNA. A siRNA may be composed of two annealed polynucleotides or a single polynucleotide that forms a hairpin structure. MicroRNAs (miRNAs) are small noncoding RNA gene products about 22 nt long that direct destruction or translational repression of their mRNA targets. Both siRNAs and miRNAs can be produced by chemical synthesis or by a living host cell in vivo or in vitro. If the complementarity between the RNA effector and the target mRNA effector binding site is partial, then translation of the target mRNA is typically repressed, whereas if complementarity is extensive, the target mRNA is typically cleaved.

In one embodiment, the effector molecule is assembled from two oligonucleotide fragments, wherein one fragment comprises nucleotide sequence of the antisense strand of the effector molecule and a second fragment comprises nucleotide sequence of the sense region of the effector molecule. In another embodiment, the sense strand is connected to the antisense strand via a linker molecule, such as a polynucleotide linker (as in a hairpin polynucleotide) or a non-nucleotide linker.

An effector may be polymerized in vitro, may be recombinant RNA, contain chimeric sequences, or may be derivatives of these groups. The RNA effector may contain ribonucleotides, deoxyribonucleotides, synthetic nucleotides, modified nucleotides, or any suitable combination such that the expression of the transgene is inhibited. The RNA effector molecules of the invention can be chemically modified. Chemically synthesized RNA effectors may contain modifications both in their polymer backbone and/or in their nucleotide bases. Polynucleotides can be synthesized using any known technique in the art. RNA effector molecules may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Different backbone linkages may be present in a single RNA effector. Bases include purines and pyrimidines, which further include the natural compounds adenine, thymine, guanine, cytosine, uracil, inosine, and natural analogs. Synthetic derivatives of purines and pyrimidines include, but are not limited to, modifications that place new reactive groups such as, but not limited to, amines, alcohols, thiols, carboxylates, and alkylhalides. The term base encompasses any of the known base analogs of DNA and RNA. The use of chemically modified RNA effectors can improve various properties of the effector molecules through increased resistance to degradation, improved cellular uptake, or enhanced activity. The use of modified nucleotides can also lengthen or shorten the half-life of the polynucleotide relative to an unmodified polynucleotide. Certain chemical modifications, (e.g. 2′-fluoro, 2′-OMe or 2′-deoxy sugars and terminus-capping chemistries), can improve the in vivo stability of synthetic RNA effector molecules and several modifications have been shown to have no or minimal effect on gene silencing activity (Czaudema et al. 2003, Layzer et al. 2004, Dande et al. 2006). Other modifications include, but are not limited to, the covalent attachment of fluorochromes for tracking purposes, or the permanent or reversible attachment of a synthetic polynucleotide to carrier molecules and/or to targeting ligands that can facilitate their delivery. Modifications can improve delivery by affecting the bioavailability of nucleic acid. Modification can enable targeting of particular cells or tissues. Many modifications are known in the art, and the present invention is not limited to any particular modification except that the effector retains the ability to mediate RNA interference. The invention is not limited to any specific modified or unmodified RNA effector.

An effector molecule may comprise a naturally occurring nucleic acid sequence or may comprise artificial, designed sequences. The RNA effector administered to the host organism may also be derived from a different species than the host. A delivered RNA effector can be either partially or perfectly complementary to effector binding sequences in the transgene mRNA translated or untranslated region. The degree of complementarity may have an effect on the molecular mechanisms of inhibition, but the invention is not limited to any of those particular molecular pathways.

The term complementarity refers to the ability of a polynucleotide to form hydrogen bond(s) with another polynucleotide sequence by either traditional Watson-Crick or other non-traditional types. In reference to the polynucleotide molecules of the present invention, the binding free energy for a polynucleotide molecule with its target (effector binding site) or complementary sequence is sufficient to allow the relevant function of the polynucleotide to proceed, e.g., enzymatic mRNA cleavage or translation inhibition. Determination of binding free energies for nucleic acid molecules is well known in the art (Frier et al. 1986, Turner et al. 1987). A percent complementarity indicates the percentage of contiguous residues in a polynucleotide molecule which can form hydrogen bonds (e.g., Watson-Crick base pairing) with a second polynucleotide sequence (e.g., 5, 6, 7, 8, 9, 10 out of 10 being 50%, 60%, 70%, 80%, 90%, and 100% complementary). “Perfectly complementary” means that all the contiguous residues of a polynucleotide sequence will hydrogen bond with the same number of contiguous residues in a second polynucleotide sequence.

Lists of known miRNA sequences can be found in databases maintained by research organizations such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature.

RNAi molecules are readily designed and produced by technologies known in the art. In addition, there are computational tools that increase the chance of finding effective and specific sequence motifs (Pei et al. 2006, Reynolds et al. 2004, Khvorova et al. 2003, Schwarz et al. 2003, Ui-Tei et al. 2004, Heale et al. 2005, Chalk et al. 2004, Amarzguioui et al. 2004).

As used herein, endogenous miRNAs are those miRNAs which are encoded in the genome of a cell. As used herein, synthetic miRNAs are those miRNAs which are not naturally produced by the cell of interest. An artificial RNA effector contains nucleotide sequence which is not naturally found in the transgene, target cell, or organism. A synthetic RNA effector can contain naturally occurring sequence or artificial sequence. Because production of endogenous miRNAs can be regulated in a tissue and developmental stage dependent manner, these miRNAs can be used to control expression of a transgene in a tissue- and developmental-stage dependent manner.

### Synthetic RNA Effector Expression Vectors

Expression vectors for generating RNA effector molecules (including but not limited to siRNA and miRNA) have been developed. These expression vectors produce the effector RNAs inside the target cell using the host cell's endogenous transcription machinery. RNA Polymerase III (Pol III) promoter-based expression vectors, including those using U6 and H1 promoters, are ideally suitable for such expression vectors (Wooddell et al. 2005, US Patent Publication 20050196862, incorporated herein by reference).

A polynucleotide containing an RNA effector expression cassette may contain a single expression unit or multiple expression units. These expression vectors can produce short hairpin structures that are similar to endogenous mature miRNA. The RNA effector expression cassette may carry two separate expression cassettes that generate two short (˜21 nucleotides), linear, complementary siRNA fragments that anneal to form an active, double stranded siRNA. In still another embodiment, the two expression cassettes expressing two ˜21-nucleotide RNA strands may reside on two separate polynucleotide vectors that are co-delivered into the host. Alternatively, the RNA effector expression cassette may encode a longer (˜50 nucleotide) RNA that folds into a short hairpin (sh) structure. This shRNA can be processed into active RNA effector duplexes by the host cell's endogenous enzymatic machinery, including the enzyme Dicer. RNA effector expression cassettes can also produce longer RNAs that are processed in the cell by the multi-step process that is characteristic of natural miRNA or siRNA maturation. By way of example, RNA effector expression cassettes can express stem-loop pri-miRNA precursors (such as those based on the naturally occurring miR30 or miR155 miRNA precursor sequence) into which artificial coding sequence can be engineered (Zeng et al. 2005, Chung et al. 2006). The resulting transcripts resemble native pri-miRNAs, and independently of the inserted sequence motif, are processed through the same cascade of events as endogenous miRNAs. SiRNAs and miRNAs produced by the multi-step process from longer transcripts may provide more potent inhibitors than the production of short hairpins or linear ˜22-mers (Dickins et al. 2005, Silva et al. 2005). In direct comparison, mature ˜22-mer RNA duplexes processed from pri-miRNA precursors showed improved silencing activity over RNA effectors expressed and processed from short hairpin precursors (Dickins et al. 2005, Silva et al. 2005). These expression cassettes have been shown to work with both Pol III and Pol II promoters, making tissue and developmental specific, in addition to constitutive expression, possible (Dickins et al. 2005, Zeng et al. 2005, Wiznerowicz et al. 2006). The regulatory effects can persist for as long as the RNA effectors are transcribed from the expression cassettes.

An RNA effector expression cassette can be carried on a circular plasmid DNA molecule that is grown in, and purified from, bacterial cells or it can be on a linear DNA. Linear DNAs can be generated by polymerase chain reaction using a template that contains the expression cassette using methods commonly available in the art. An expression cassette may comprise any or all of the following elements: promoter, transcriptional start site, coding region for the effector RNA, transcription terminator, intron, enhancer, and polyadenylation signal. The design of expression cassettes is well known in the art. The promoter element of the siRNA expression cassette can be selected from the group comprising: RNA polymerase III (Pol III) promoters, RNA polymerase II (Pol II) promoters, which may originate from various species (US Patent Publication No. 20050196862, incorporated herein by reference). Pol III promoters may be selected from the list comprising: U6 promoters, H1 promoters, and tRNA promoters. Pol III initiates RNA synthesis at a well-defined distance from the promoter and terminates when a string of 4-5 uridines is encountered. Pol II promoters may be selected from the list comprising: U1, U2, U4, and U5 promoters, snRNA promoters, microRNA promoters, and mRNA promoters. In one embodiment, the effector expression cassette contains a Pol II liver-specific long term expression promoter (U.S. patent application Ser. No. 10/229,786, incorporated herein by reference). RNA effector cassettes can be delivered to a cell wherein the DNA is transcribed to produce hairpin RNAs or separate sense and anti-sense strand linear RNAs. The RNA effector expression cassettes may also contain sequences that do not serve a specific function in the target cell but are used in the generation of the polynucleotide. Such sequences include, but are not limited to, sequences required for replication in bacterial host cells or selection of the polynucleotide in a host organism, such as antibiotic resistance genes and their promoters. An expression cassette may also include sequences which allow replication of the polynucleotide in mammalian cells, or which facilitate its integration into the host cell's genome (e.g. a transposon).

### Effector Binding Site

An effector binding site is a nucleotide sequence which is complementary or partially complementary to at least a portion of an effector. An RNA effector can be an endogenous RNA effector, such as a miRNA, or a delivered synthetic or artificial RNA effector. The sequence can be a perfect match, meaning that the binding site sequence has perfect complementarity to the effector. Alternatively, the sequence can be partially complementary, meaning that one or more mismatches may occur when the effector is base paired to the binding site. For miRNA effectors, partially complementary binding sites preferably contain perfect or near perfect complementarity to the seed region of the miRNA. The seed region of the miRNA consists of the 5′ region of the miRNA from about nucleotide 2 to about nucleotide 8. For naturally occurring miRNAs and target genes, miRNAs with perfect complementarity to an mRNA sequence direct degradation of the mRNA through the RNA interference pathway while miRNAs with imperfect complementarity to the target mRNA direct translational control (inhibition) of the mRNA. Similarly, siRNAs with perfect complementarity to an mRNA sequence generally direct degradation of the mRNA through the RNA interference pathway while siRNAs with imperfect complementarity to the target mRNA generally direct inhibition of translation of the mRNA. The invention is not limited by which pathway, degradation or inhibition, is utilized by the effector in inhibiting expression of the gene. In a preferred embodiment, the effector binding site is located in the 3′ untranslated region (UTR) of the gene mRNA. In another embodiment, the effector binding site(s) are positioned just downstream of a 3′ UTR intron and about 100 nucleotides upstream of a polyadenylation signal. However, as is noted in the examples, other sites are permissible.

As used herein, cognate effector and effector binding site refer to an effector and binding site that typically interact. For a given effector, a sequence to which a given RNA effector binds with sufficient affinity to induce RNA interference, is the cognate effector binding site for the RNA effector. Similarly, for a given effector binding site, an effector which binds to the sequence with sufficient affinity to induce RNA interference, is the cognate effector for the effector binding site. For a given nucleotide sequence, a cognate RNA effector which binds to the sequence is readily generated using methods standard in the RNA interference art. Conversely, for a given RNA effector nucleotide sequence, an effector binding site sequence is readily generated.

Incorporating effector binding sites into transgene expression vectors provides tools to control transgene expression. The transgene expression cassette can be designed such that the transcript contains one or more effector binding site(s) for its cognate RNA effector. The effector binding sites can be sequences which complementary to RNA effectors naturally present in the cell or they can be artificial. As used herein, an ectopic effector binding site is a nucleotide sequence which does not naturally occur in the gene of interest. Expression of the transgene is then regulated either by the cell's natural environment (by an endogenous, naturally occurring miRNA), or by the delivery of an exogenous effector, such as a synthetic RNA effector.

The presence of a single, perfectly matched effector binding site in the transcribed mRNA of the transgene may be sufficient to inhibit expression of the transgene in cells in which the cognate RNA effector is present. However, the invention is not limited to expression cassettes containing a simple perfectly matched effector binding site. A transgene expression cassette of the invention may contain one or more RNA effector binding sites with perfect or imperfect complementarity (imperfect effector binding sites). A transgene expression cassette may also contain both perfect and imperfect effector binding sites. Transgene expression cassettes can be tailored to result in varying levels of regulation by using single perfect, multiple perfect, single imperfect, multiple imperfect or a combination of perfect and imperfect effector binding sites. Further, effector binding sites for different cognate RNA effectors may also be used, thereby permitting a gene to be regulated by multiple effectors. A preferred location for the effector binding site is the 3′UTR. However, effector binding site sequences inserted into either coding or 5′-UTR sequences may also be used. SiRNA effectors typically bind to target sites inside the protein coding region.

The transgene can be any gene which encodes a protein of interest and includes both therapeutic genes and genes of biological interest, such as for biological research. The gene can be endogenous to the species, a foreign gene, or a recombinant gene. The transgene is meant to include a gene whose expression in a cell effects the biological properties of the cell, tissue or organism. The transgene comprises a gene for which the sequence is known or can be known and which is delivered to a cell in vivo or in vitro. The nucleic acid carrying the transgene may be extrachromosomal (e.g. a plasmid DNA or a cosmid) or may be integrated into the host cell's chromosome (e.g. on a transposon, such as Sleeping Beauty).

The term gene generally refers to a nucleic acid sequence that comprises coding sequences necessary for the production of a nucleic acid (e.g., RNA) or a polypeptide (protein) or protein precursor. A polypeptide can be encoded by a full-length coding sequence or by any portion of the coding sequence so long as the desired activity or functional properties (e.g., enzymatic activity, ligand binding, signal transduction) of the full-length polypeptide or fragment are retained. In addition to the coding sequence, the term gene may also include, in proper contexts, the sequences located adjacent to the coding region on both the 5′ and 3′ ends which correspond to the full-length mRNA (the transcribed sequence) or all the sequences that make up the coding sequence, transcribed sequence and regulatory sequences. The sequences that are located 5′ or upstream of the coding region and which are present on the mRNA are referred to as 5′ untranslated region (5′ UTR). The sequences that are located 3′ or downstream of the coding region and which are present on the mRNA are referred to as 3′ untranslated region (3′ UTR). The term gene encompasses synthetic, recombinant, cDNA and genomic forms of a gene. A genomic form of a gene contains the coding region interrupted with non-coding sequences termed introns, intervening regions or intervening sequences. Introns are segments of a gene, which are transcribed into nuclear RNA. Introns may contain regulatory elements such as enhancers, or they may encode regulatory elements such as miRNAs. Introns are removed or spliced out from the nuclear or primary transcript; introns therefore are absent in the mature mRNA transcript. Regulatory sequences in a gene include, but are not limited to, promoters, enhancers, transcription factor binding sites, polyadenylation signals, internal ribosome entry sites, silencers, insulating sequences, matrix attachment regions. Non-coding sequences may influence the level or rate of transcription and/or translation of the gene, and they may also influence mRNA stability. Sequence motifs in non-coding regions of the gene are also subject to effector-mediated regulation. In one embodiment, the level of transgene can be controlled by controlling gene dosage. Controlling gene dosage can be accomplished by repetitive delivery of smaller transgene doses. By providing multiple small doses, the treatment can be halted when the correct expression level is achieved.

Transgene expression cassettes of the described invention contain one or more effector binding sites within their transcribed RNA that are recognized by cognate RNA effectors. The invention is not limited by the location of the binding site(s) within the transcript. Reporter genes can be used to test the efficacy of an effector binding site or of a given expression cassette. The cassette is tested by substituting the reporter gene for the transgene. Reporter genes include luciferases, fluorescent proteins such as green fluorescent protein, β-galactosidase, chloramphenicol acetyl transferase, secreted alkaline phosphatase, and the like. Expression of the transgene from the transgene expression cassettes and regulation of protein production by the cognate effector may be validated either in vitro or in vivo.

The term polynucleotide, or nucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications that place new reactive groups such as, but not limited to, amines, alcohols, thiols, carboxylates, and alkylhalides. The term base encompasses any of the known base analogs of DNA and RNA. The term polynucleotide includes deoxyribonucleic acid (DNA) and ribonucleic acid (RNA) and combinations on DNA, RNA and other natural and synthetic nucleotides. Expression vectors carrying miRNA binding sites both for the purpose of regulating expression levels and for studying miRNA expression and function have been described (U.S. Patent Publication 20070054872, incorporated herein by reference; U.S. Patent Publication 20060265771, incorporated herein by reference).

By inhibit it is meant that the expression of the gene, or level of RNAs, encoding one or more protein subunits, or activity of one or more protein subunits, such as hormones, growth factors, enzymes, pathogenic protein, viral protein or cancer related protein subunit(s), is reduced below that observed in the absence of the compounds or combination of compounds of the invention. In one embodiment, inhibition or down-regulation with an RNA effector molecule preferably is below that level observed in the presence of a control inactive RNA effector molecule. A control RNA effector molecule includes an oligonucleotide with scrambled sequence or with mismatches such that it does not bind to the effector binding sequence.

Delivery of the RNA effector or the transgene is not limited to any particular delivery method. The effector or transgene can be delivered to cells in a mammal using gene delivery methods practiced in the art. Known nucleic acid delivery methods include, but are not limited to: hydrodynamic injection (U.S. Pat. Nos. 6,265,387 and 6,627,616, both incorporated herein by reference; Liu et al. 1999; Zhang et al. 1999), hydrodynamic limb vein (HLV) injections (U.S. Patent Publication 20040242528, incorporated herein by reference), direct parenchymal injection, biolistic transfection, lipid transfection (lipofection), polycation mediated transfection (polyfection), lipid-polycation complex mediated transfection (lipopolyfection), chemical-mediated transport, viral delivery, and electroporation (Cemazar et al. 2006). The effector may be introduced along with components that perform one or more of the following activities: enhance uptake by the cell, promote annealing of the duplex strands, stabilize the annealed strands, or other-wise increase inhibition of the target gene. Preferably, the delivery methods allow repeated administration of polynucleotides into the same cells. When identifying, screening and validating siRNA and/or miRNA sequence motifs in an in vitro setting prior to their in vivo use, conventional transfection procedures can be used.

As used herein, co-delivery of the effector molecule with the transgene means simultaneous delivery, delivery within 0 to 24 hours, or delivery within 1-3 days. Co-delivery can be used to attenuate a high initial transgene expression peak that can occur with transgene delivery in vivo.

Medical or biomedical research applications of the invention include, by way of example, the knockdown of too high EPO expression in mammals treated for anemia by gene therapy. Over-expression of EPO leads to abnormal levels of erythropoiesis. As a result, the mammal develops elevated hematocrit and the cellular components of the blood can reach a level that causes high viscosity and an increased tendency to form blood clots. This pathological state—called polycythemia—can lead to thrombotic complications, hemorrhages, stroke or heart attack. While phlebotomy can provide temporary relief by diluting the cellular components of the blood, down-regulation of EPO transgene expression provides a better, longer-lasting solution. Additional examples of uses include, but are not limited to, silencing of toxic or suicide genes delivered as a treatment for cancer, or attenuating the overexpression of hormones, growth factors, coagulation factors, recombinant therapeutic antibodies, immunomodulators etc.

The described transgene regulation method can also be used in model organisms, such as laboratory animals, that are used to develop model systems for clinical gene therapy, or are used to answer basic scientific questions. Thus, the term “patient” and “host” can be used interchangeably and include both human and non-human species and their tissues or cells. Any organ, tissue or cell type of the host can be the target of the gene regulation disclosed in this invention. Preferably, repeat delivery of polynucleotides to the cell or tissue is possible. Furthermore, the invention can also be used in cells derived from human or non-human hosts, which are in an in vitro state, cultured outside of the body. Testing of RNA effectors and transgene expression constructs containing RNA effector binding sites can be performed in in vitro cultured host cells.

While RNA effectors are most commonly used to inhibit gene expression, RNA effectors can also, indirectly, up-regulate expression of a secondary target through inhibition of an inhibitor.

### EXAMPLES

### Example 1

Human EPO-specific siRNA sequences. The list of 10 sequences below represents 19-mer fragments of the human erythropoietin coding region that are potential target sequences for RNA interference. These are examples only and the effective knockdown of EPO expression described in this invention is not limited to any particular siRNA sequence on this list. Other sequence motifs in the huEPO coding region may also be suitable.

| | |
|---|---|
| siRNA | Sequence |

| | |
|---|---|
| huEPO-siRNA No. 1 | GCCGAGUCCUGGAGAGGUA, |
| | SEQ ID NO. 1 |
| | CGACGGGCUGUGCUGAACA, |
| | SEQ ID NO. 2 |
| huEPO-siRNA No. 2 | GCUGAACACUGCAGCUUGA, |
| | SEQ ID NO. 3 |
| | CUGAACACUGCAGCUUGAA, |
| | SEQ ID NO. 4 |
| huEPO-siRNA No. 3 | CUGCUCCACUCCGAACAAU, |
| | SEQ ID NO. 5 |
| | CUGACACUUUCCGCAAACU, |
| | SEQ ID NO. 6 |
| | GCAAACUCUUCCGAGUCUA, |
| | SEQ ID NO. 7 |
| huEPO-siRNA No. 4 | UCUUCCGAGUCUACUCCAA, |
| | SEQ ID NO. 8 |
| | UUCCGAGUCUACUCCAAUU, |
| | SEQ ID NO. 9 |
| | UCCGAGUCUACUCCAAUUU, |
| | SEQ ID NO. 10 |

The sequence motifs represent the coding (sense) strand of the human EPO mRNA. Oligonucleotides corresponding to the above sequences and complementary strands were synthesized and annealed to form the double strand siRNA effectors. Both the sense and the antisense strands additionally contained 3′ uridine dinucleotide overhangs.

### Example 2

Regulation of EPO expression—in vitro test of effector efficacy. Four siRNA motifs were selected from the above list of possible effector binding sites. No. 1 targeted Exon 2 of the huEPO gene, No. 2 targeted Exon 3, and Nos. 3 and 4 targeted Exon 5. Homology search against the human genome database indicated high level of sequence specificity for the EPO coding sequence for these siRNAs. The siRNAs (synthesized by Dharmacon, Lafayette, Colo.) were used as annealed, desalted RNA duplexes dissolved in 100 mM NaCl, 10 mM TrisHCl pH 7.0. An in vitro validation test was performed using HeLa cell cultures transiently transfected with a plasmid vector expressing human EPO, CMV-huEPO pDNA. The transiently transfected cells were plated into 24-well tissue culture plates at 1×10⁵ cell/well density and 24 hr later they were transfected with 1, 5, 25 or 50 nM of each siRNA using TRANSIT-TKO® transfection reagent (Mirus Bio Corp. Madison, Wis.). The huEPO-siRNAs Nos. 1-2 and Nos. 3-4 were also transfected together, pooled at 25 nM concentration each. Besides using EPO-producing control cells that remained untreated by siRNA, controls also included:
1) cells treated only with the TRANSIT-TKO® reagent at concentrations that were used for the delivery of 1, 5, 25 or 50 nM siRNA,
2) cells transfected with a luciferase-specific control siRNA (GL3-153) at 50 nM, and
3) negative control HeLa cells that were not transfected with human EPO. Three wells were used for each condition.

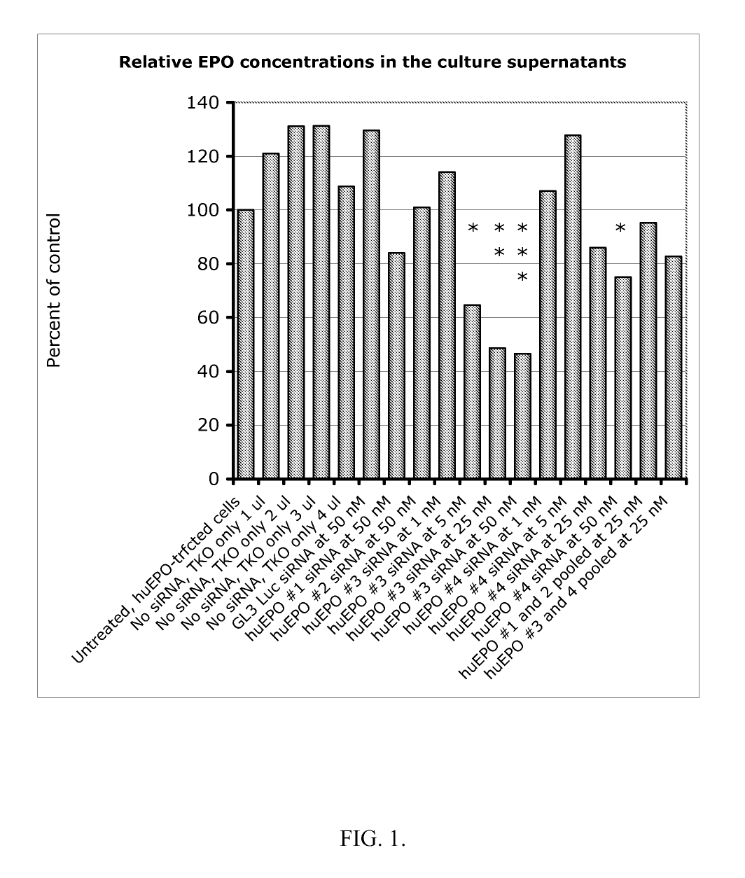

24 hr after siRNA delivery, media were changed to remove all previously produced and secreted EPO from the supernatants. After an additional 24 hr, culture supernatants were harvested and were assayed for EPO content using a huEPO ELISA Kit (R&D Systems, Minneapolis, Minn., USA). EPO production of the untreated huEPO-transfected culture was considered 100% and the effect of mock TRANSIT-TKO®, or siRNA+TRANSIT-TKO® transfection was expressed as percent EPO produced relative to the control. Negative control cells that were not transfected with the CMV-huEPO pDNA did not produce detectable EPO and are not included in the graph. The results are summarized in FIG. 1. SiRNAs No. 1 and 2 had only modest effect at 50 nM concentration. SiRNA No. 3 was the most effective, resulting in dose-dependent knockdown of expression between 5-50 nM concentration. All three concentrations resulted in significantly reduced EPO levels in the culture supernatants: for the 5 nM sample knockdown was 50.7% (p<0.05), for 25 mM it was 62.9% (p<0.01) and for the 50 mM sample it was 64.4% (p<0.001). The effect of siRNA No. 4 was significant at the highest, 50 nM concentration, with 42.8% knockdown (p<0.05).

### Example 3

In vivo delivery of huEPO-specific effectors to mouse muscle. The huEPO-siRNA No. 3 that was found to be most effective in vitro for controlling EPO expression in cultured cell validation test was also delivered in vivo in mice. ICR mice (˜25 g) were injected either with 10 μg CMV-huEPO expression plasmid alone, or were co-injected with a mixture of this plasmid and 5 μg siRNA, in 1 ml sterile, normal saline solution via the hydrodynamic limb vein delivery procedure (US Patent Publication No. 20040242528). Control animals received the pDNA mixed with 5 μg of a control siRNA (GL3-153, specific for firefly luciferase). Test animals received the pDNA with 5 μg of the huEPO-specific siRNA No. 3, which targeted Exon 5 of the huEPO mnRNA. Each group had 5 animals. Serum samples were collected on days 2, 3 and 6 post-injection, and the physiological effect of produced EPO was assessed by measuring Hematocrit (Hct) values in whole blood samples collected 10 and 14 days after delivery. Serum EPO levels were determined using the huEPO ELISA Kit, and Hct values were determined by sedimenting whole blood samples in heparin-coated capillary tubes, using standard clinical laboratory methods.

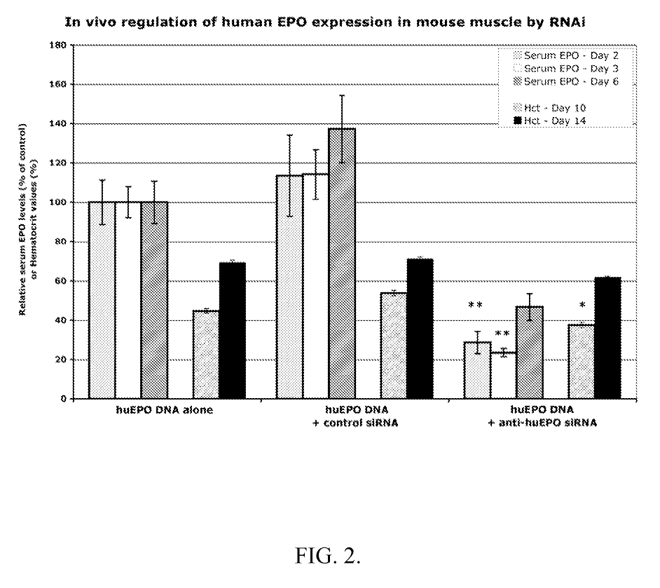

The results are summarized on FIG. 2. When huEPO-siRNA No. 3 was co-injected with the pDNA, on Day 2 and Day 3 post-injection serum EPO levels were <30% of the DNA-only control. This difference was statistically significant (p<0.005). The inhibitory effect started to decline by Day 6 post-injection resulting in an increase in serum EPO levels to ˜50% of the control. Decreased inhibition of transgene expression by day six is likely the result of RNA degradation in the target cells. Longer or shorter-term inhibitory effects may be accomplished by using modified RNA effectors or by expressing the RNA effector in the target cell in vivo from an effector expression cassette.

The co-delivery of the non-specific control siRNA with the EPO expression plasmid slightly increased serum EPO levels (up to 137% of the DNA-only group) and erythropoiesis. It is possible that synthetic siRNAs trigger some non-specific response in the myofibers that mildly up-regulates transcription. Alternatively, the presence of extra nucleic acids in the injection fluid increases the efficiency of DNA delivery. In either case, a similar effect can be expected in animals injected with the huEPO-specific siRNA. Taking this into account, the degree of knockdown in the huEPO-siRNA-injected animals may have been even more pronounced (<20-25%).

In spite of the fairly short life of the RNA effectors under these experimental conditions, these results demonstrate that even the transient down-regulation of EPO expression from the delivered plasmid resulted in significantly lower Hct values on Day 10 (p<0.05). In animals that had been co-injected with pDNA and huEPO-siRNA No. 3, erythropoiesis was not triggered at all by Day 10: Hct was at normal, <40% levels. In contrast, the group that received pDNA and control siRNA had >50% Hct values 10 days after delivery. By Day 14, the huEPO-siRNA-injected group's Hct also became elevated, but it remained lower than that of the control groups'. However, the difference was no longer significant.

### Example 4

In vivo delivery of huEPO-expressingpDNA and huEPO-specific siRNA into non-human primates.

### A. Human EPO Expression in Immunocompetent Rhesus Monkeys.

Three immunocompetent rhesus monkeys were injected with 1.3 mg pDNA/kg body weight dose of an expression vector carrying a truncated version of the huEPO gene. One of the animals also received—co-injected with the pDNA—a total of 1 mg unmodified, synthetic negative control siRNA that was specific for human secreted alkaline phophatase (huSEAP). The third animal was co-injected with the pDNA and 1 mg (total) unmodified, synthetic huEPO-siRNA No. 3. The injections were performed using the hydrodynamic limb vein procedure as described (US Patent Publication 20040242528; Sebestyen et al. 2007). Blood and serum samples were collected more than a week before gene delivery (to record baseline values), and 2, 6, 9, 14, 28, 42, 56 and 63 days after the procedure. Serum samples were analyzed for their EPO concentration using huEPO ELISA (R&D Systems). Blood samples were analyzed for complete blood counts and reticulocyte counts at a clinical laboratory (Meriter Hospital, Madison, Wis.).

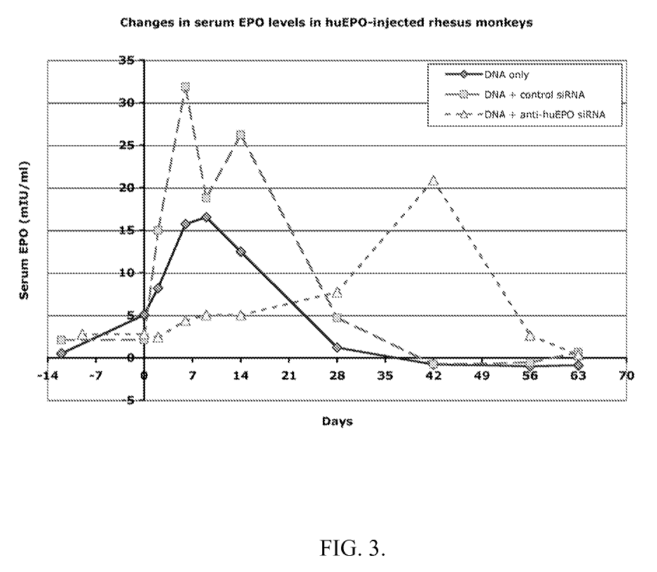

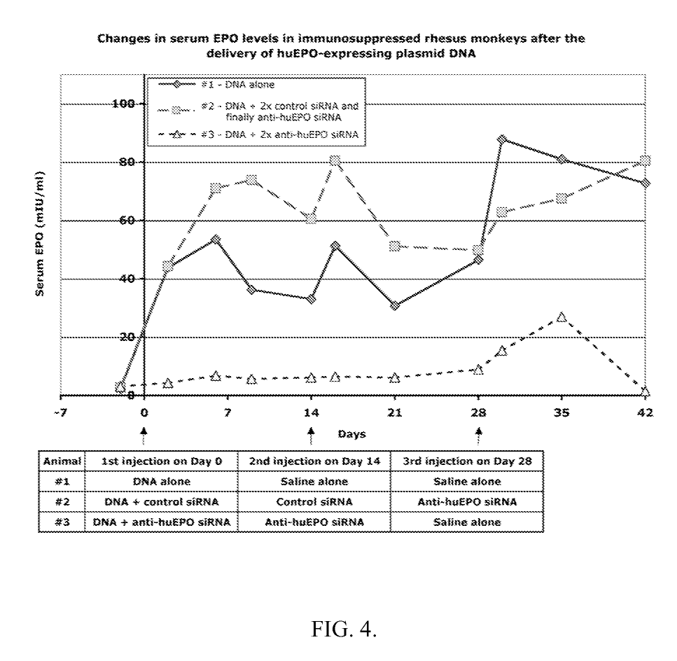

Results are summarized on FIG. 3. The two animals that received pDNA either alone or mixed with the huSEAP-specific control siRNA showed elevated serum EPO levels 2 days post-injection. EPO concentrations continued to increase during the first 2 weeks of the study, with some fluctuation. At all time points during the first 4 weeks, serum EPO levels in the animal that also received control siRNA exceeded those of the animal injected with pDNA only. The animal injected with the pDNA+huEPO-siRNA mixture showed significantly lower serum EPO levels: 14-27% of those in the “pDNA+control siRNA” animal, and 28-40% of those in the “pDNA-only” animal at time points between Days 2-14. After Day 14, EPO expression increased in the huEPO-siRNA-treated animal, likely resulting from degradation or loss of the RNA effector. Expression peaked on Day 42 post-injection (20.1 mIU/ml). For the pDNA-only animal, transgene expression peaked on day 9 (16.5 mIU/ml).

Since the animals were immunocompetent and the expressed protein was the human EPO ortholog, serum EPO levels dropped dramatically as soon as the animals mounted an immune response to the transgene. The immune response started after Day 14 in the animals that started to express immediately after gene delivery. Immune response was delayed until after Day 42 in the huEPO-siRNA-treated animal. By 9 weeks after gene delivery none of the animals had detectable serum EPO concentration.

B. Human EPO expression in immunosuppressed rhesus monkeys. Due to the rapid immune rejection of the human EPO ortholog, immunosuppressed primates were tested to provide a longer observation period. A higher pDNA transgene dose was also used. Three rhesus monkeys were treated with 2 mg/kg prednisolone (Solu-Delta-Cortef®, Pharmacia & Upjohn, Kalamazoo, Mich.) started 24 hr prior to gene delivery and given daily as an intramuscular injection for 14 days. The prednisolone dose was then reduced to 1 mg/kg for 4 weeks, followed by gradual tapering of the treatment to 0.5 and 0.2 mg/kg doses. Baseline serum EPO levels and hematological parameters were also recorded prior to gene delivery. The animals were then injected, on day 0, with 2.6 mg/kg dose of huEPO-expressing pDNA either alone (animal #1), or mixed with 1 mg control (huSEAP-specific; animal #2) or huEPO-specific (animal #3) siRNA, using the hydrodynamic limb vein procedure. Repeat deliveries were performed on days 14 and 28 after the initial delivery as illustrated on FIG. 4. The same limb was targeted each time, using the same vein as on Day 0. On day 14, animal #1 received empty saline solution, while animals #2 and #3 received a second 1 mg (total) dose of the control or huEPO-specific siRNA. On day 21, huEPO-siRNA was delivered to a single animal that had shown the highest EPO expression level and the highest hematocrit values: animal #2. Serum and blood samples were collected at several time points and were analyzed as described for Example 4A.

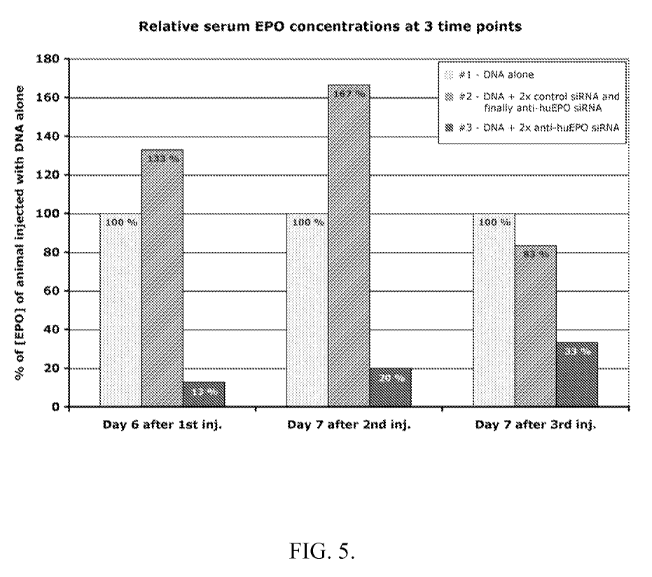

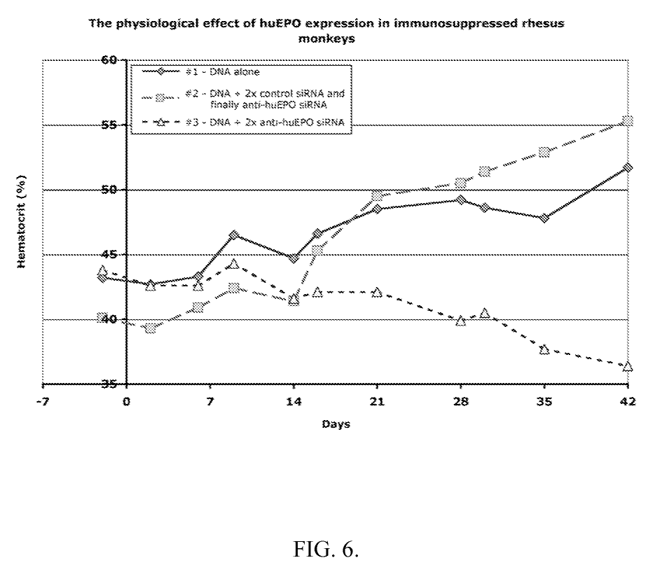

Results are shown on FIGS. 4-6. During the first 4 weeks of the study, animals #1 and #2 showed highly elevated serum EPO levels compared to pre-injection baselines and compared to animal #3, which received the huEPO-siRNA (FIG. 4). Both animals responded to the EPO production by increased erythropoiesis, leading to hematocrit values of ˜50% by the end of the 4^{th} week (FIG. 6). In contrast, animal #3 had serum EPO concentrations barely above baseline and showed a mild decrease in Hct values (FIGS. 4 and 6). During the first 4 weeks of the study, in the animal injected twice with the EPO-specific siRNA, up to 92% inhibition of expression was achieved relative to the animal injected twice with the control siRNA (specific for huSEAP), and inhibition between 80-90% was recorded relative to the animal that received DNA alone. Similarly to the study described for Example 4A, the animal co-injected with pDNA+control siRNA (animal #2) expressed more EPO (between 133-204%) and had more pronounced physiological response than the animal receiving pDNA alone (animal #1; FIG. 6). As illustrated on FIG. 5, 7 days after the 3rd injection, huEPO-siRNA caused decreased serum EPO concentration in animal #2 to only 83% relative to that of animal #1: a 2-fold drop from the 167% value two weeks earlier. At the same time, serum EPO concentration in animal #3 started to increase, as the effect of earlier huEPO-siRNA injections diminished. These results demonstrate that expression from a transgene could be attenuated by delivering an siRNA effector. As illustrated on FIG. 4, the injection procedure caused a temporary spike in serum EPO levels, possibly caused by releasing intracellular proteins.

### Example 5

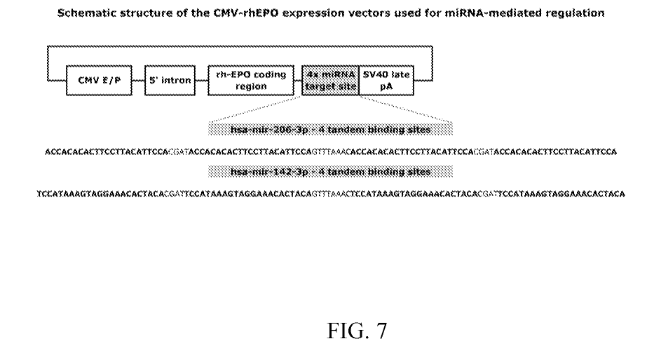

Construction of a rhesus EPO expression vector containing ectopic miRNA binding sites. A plasmid vector containing the rhesus EPO (rhEPO) cDNA in an expression cassette comprising the CMV immediate/early enhancer and promoter, a β-globin-IgG hybrid intron and the late SV40 polyadenylation region (Sebestyen et al. 2007) was modified by adding ectopic hsa-mir-206-3p or hsa-mir-142-3p miRNA binding sites into the 3′UTR of the transgene. First a unique restriction site was introduced downstream of the rhEPO stop codon and upstream of the polyA signal. This vector was used as one of the controls for the in vivo study presented in Example 6. The new restriction site was then used to insert a 108 bp synthetic oligonucleotide fragment containing 4 identical, tandem copies of miRNA effector binding sites either for the skeletal muscle-specific hsa-mir-206-3p miRNA (Kim et al. 2006), or for hsa-mir-142-3p, which is expressed in cells of hematopoietic origin (Chen et al. 2004). The target sites were designed to be perfectly complementary to the known muscle-expressed miRNAs. MiRNA binding sites for other known miRNAs can be inserted into other expression vectors for delivery to muscle or other tissues. The schematic structure of the plasmids and the sequence of the synthetic inserts are illustrated on FIG. 7.

### Example 6

miRNA-regulated EPO expression in mouse muscle. To assess the effect of miRNA binding on rhEPO expression in mouse muscle, four pDNA constructs were delivered into the limb of ICR mice via hydrodynamic limb vein injections. Animals (n=4 mice/group) were injected with 20 μg pDNA/animal using the constructs containing four ectopic hsa-mir-206-3p miRNA effector binding sites (shown in FIG. 7). Control groups were injected with 20 μg pDNA/animal of:
a) plasmid that did not contain any protein-coding region (non-expressing pDNA control),
b) pCMV-RhEPO construct without ectopic miRNA-target sites, or
c) RhEPO construct containing four ectopic hsa-mir-142-3p miRNA binding sites. 142-3p miRNA is not expressed in skeletal muscle.

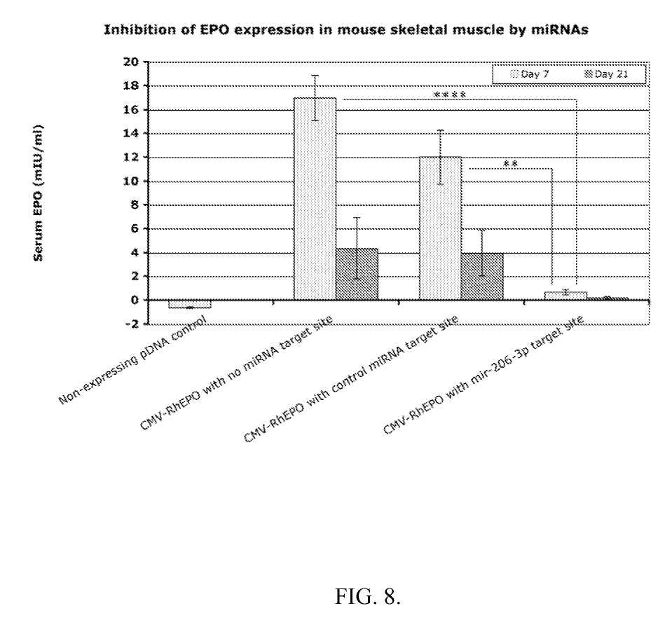

Serum samples were collected 7 and 21 days post-injection to determine serum EPO concentrations, and blood samples were collected 14 days after delivery to determine the animals' hematocrit values. EPO concentrations were assessed using the human EPO-specific ELISA kit, which cross-reacts with the rhesus ortholog (R&D Systems). The antibody in the kit has lower avidity for rhEPO than for the huEPO used as a standard, leading to an approximately 4-fold under-estimation of actual EPO concentrations (Rivera et al. 2005). The graph in FIG. 8. shows the original ELISA readings without correcting them for this 4-fold discrepancy. Hct values were determined as described above for Example 3.

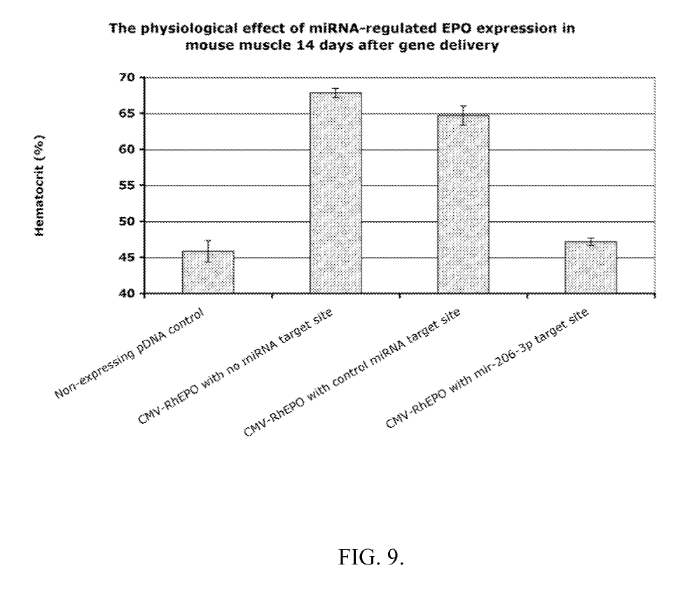

The results are summarized on FIG. 8-9. Serum EPO levels on Day 7 clearly demonstrated that the insertion of the hsa-mir-206-3p miRNA binding sites into the 3′UTR of the rhEPO transcript dramatically reduced EPO expression in cells where this miRNA is abundant (FIG. 8). When compared to the group that had received CMV-RhEPO with no cognate effector binding sites, or to the group that was injected with the construct carrying the control hsa-mir-142-3p binding sites, the difference between serum EPO levels were highly significant (p<0.0001 or p<0.005, respectively). Expression levels from the control hsa-mir-142-3p binding site construct relative to the parental empty vector were not statistically significant (p=0.14). The almost complete inhibition of EPO expression from the hsa-mir-206-3p construct illustrates that properly designed transcripts are targets for miRNA effector-mediated down-regulation. By Day 21 post-injection, some animals in each group had mounted an immune response against the foreign rhEPO ortholog (FIG. 8).

The physiological response to the produced rhEPO also reflected the regulatory effect of the hsa-mir-206-3p miRNAs. Two weeks after DNA delivery, the average Hct value of mice injected with the construct carrying hsa-mir-206-3p binding sites remained very close to Hct values in the group injected with the non-expressing control pDNA (p=0.15; FIG. 9). In contrast, Hct in the two other groups treated with the construct carrying either hsa-mir-142-3p binding sites or no binding sites at all, increased dramatically, from ˜45% to ˜65% (p<10⁻⁵).

These results demonstrate that the insertion of miRNA effector binding sites into the 3′UTR of the EPO transcript provides means for the miRNA-mediated regulation of transgene expression. Effector binding sites can be further engineered to be responsive to synthetic or artificial miRNA sequences.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

We claim:

1. A method regulating transgene expression in vivo comprising: delivering an RNA effector molecule to the cell in which the transgene is expressed wherein the RNA effector molecule contains sequence that is complementary to a sequence present in a messenger RNA transcribed from the transgene.

2. The method of claim 1 wherein the RNA effector molecule comprises: synthetic RNA interference-inducing polynucleotide, small interfering RNA, microRNA, small hairpin RNA, double strand RNA, and RNA effector expression cassette.

3. The method of claim 2 wherein the RNA effector molecule contains natural or artificial sequence.

4. The method of claim 3 wherein the RNA effector molecule contains complete or partial complementarity to the sequence present in the messenger RNA transcribed from the gene.

5. The method of claim 4 wherein the RNA effector molecule causes degradation of the transgene messenger RNA.

6. The method of claim 4 wherein the RNA effector molecule causes inhibition of translation of the transgene messenger RNA.

7. The method of claim 4 wherein the transgene comprises a therapeutic gene.

8. The method of claim 7 wherein the therapeutic gene comprises an erythropoietin gene.

9. A method for regulating transgene expression in vivo comprising:
a) forming a transgene expression cassette wherein the expression cassette contains an RNA effector binding site for a cognate RNA effector molecule wherein the RNA effector binding site is present on a messenger RNA transcribed from the transgene expression cassette and
b) delivering the transgene to a cell in vivo.

10. The method of claim 9 wherein the RNA effector binding site consists of an ectopic RNA effector binding site.

11. The method of claim 9 wherein the RNA effector binding site consists of an artificial sequence or a naturally occurring sequence.

12. The method of claim 9 wherein the RNA effector binding site is located in a 3′ untranslated region of the messenger RNA.

13. The method of claim 9 wherein the cognate RNA effector molecule consists of a naturally occurring microRNA present in the cell.

14. The method of claim 9 further comprising delivering the cognate RNA effector molecule to the cell.

15. The method of claim 14 wherein the cognate RNA effector molecule comprises: synthetic RNA interference-inducing polynucleotide, small interfering RNA, microRNA, small hairpin RNA, double strand RNA, and RNA effector expression cassette.

16. The method of claim 15 wherein the RNA effector molecule contains natural or artificial sequence.

17. The method of claim 16 wherein the RNA effector molecule contains complete or partial complementarity to the RNA effector binding site in the messenger RNA transcribed from the transgene.

18. The method of claim 9 wherein the cognate RNA effector molecule causes decreased expression of the transgene.

19. The method of claim 18 wherein the transgene comprises a therapeutic gene.

20. The method of claim 19 wherein the therapeutic gene comprises an erythropoietin gene.

21. The method of claim 20 wherein decreased expression of the erythropoietin gene is used to treat or prevent polycythemia associated with overexpression of the erythropoietin gene.

22. A transgene expression cassette comprising: a promoter, a coding sequence for a transgene, and one or more ectopic RNA effector binding sites.

23. The transgene expression cassette of claim 22 wherein the transgene further contains one or more elements selected from the list consisting of: polyadenylation signal, 3′ untranslated region, 5′ untranslated region, intron, and enhancer.

24. The transgene expression cassette of claim 22 wherein the RNA effector binding site is located in a messenger RNA transcribed from the expression cassette.

25. The transgene expression cassette of claim 22 wherein the RNA effector binding site is located in the 3′ untranslated region, 5′ untranslated region, or a protein coding region of the messenger RNA.

26. The transgene expression cassette of claim 22 wherein the RNA effector binding site consists of an artificial sequence or a naturally occurring sequence.

27. The transgene expression cassette of claim 26 wherein the RNA effector binding site consists of a cognate RNA effector binding site for a naturally occurring microRNA.

28. The transgene expression cassette of claim 26 wherein the RNA effector binding site consists of a cognate RNA effector binding site for a synthetic RNA effector molecule or artificial RNA effector molecule.

29. The transgene expression cassette of claim 22 wherein the transgene comprises a therapeutic gene.

30. The transgene expression cassette of claim 29 wherein the therapeutic gene comprises an erythropoietin gene.


SEQUENCE LISTING (10 sequences)

SEQ ID NO: 1 - RNA, 19 nt, Homo sapiens
gccgaguccuggagaggua

SEQ ID NO: 2 - RNA, 19 nt, Homo sapiens
cgacgggcugugcugaaca

SEQ ID NO: 3 - RNA, 19 nt, Homo sapiens
gcugaacacugcagcuuga

SEQ ID NO: 4 - RNA, 19 nt, Homo sapiens
cugaacacugcagcuugaa

SEQ ID NO: 5 - RNA, 19 nt, Homo sapiens
cugcuccacuccgaacaau

SEQ ID NO: 6 - RNA, 19 nt, Homo sapiens
cugacacuuuccgcaaacu

SEQ ID NO: 7 - RNA, 19 nt, Homo sapiens
gcaaacucuuccgagucua

SEQ ID NO: 8 - RNA, 19 nt, Homo sapiens
ucuuccgagucuacuccaa

SEQ ID NO: 9 - RNA, 19 nt, Homo sapiens
uuccgagucuacuccaauu

SEQ ID NO: 10 - RNA, 19 nt, Homo sapiens
uccgagucuacuccaauuu